(19) United States
(12) Patent Application Publication
(10) Pub. No.: US 2010/0178311 A1
(43) Pub. Date: Jul. 15, 2010

(54) IMPLANT AND METHOD FOR ITS MANUFACTURE

(76) Inventors: Stephan Barcikowski, Hannover (DE); Andreas Schuessler, Pfinztal (DE)

Correspondence Address: FLYNN THIEL BOUTELL & TANIS, P.C., 2026 RAMBLING ROAD, KALAMAZOO, MI, 49008-1631, US

(21) Appl. No.: 12/452,335

(22) PCT Filed: Jun. 27, 2008
(86) PCT No.: PCT/EP2008/005288

§ 371 Date: Mar. 4, 2010

(30) Foreign Application Priority Data
Jun. 27, 2007 (DE) 10 2007 029 672.1

(51) Int. Cl. A61F 2/82; C25D 13/10; C25D 13/12

(52) U.S. Cl. 424/423; 204/471

(57) ABSTRACT

The invention is directed to an implant (1) with a main body (2) having a surface (5,6), and with a coating (7) of nanoparticles (8) provided at least on portions of the surface (5,6) of the main body (2), wherein the main body (2) is made from a material with a metal lattice structure, and the nanoparticles (8) of the coating (7) comprise a material which also has a metal lattice structure. The implant is characterized by the lattice structure of the material of the nanoparticles (8) being compatible in such a way to the lattice structure of the material of the main body (2), that both materials are mutually connectable by a diffusion process, in particular by a diffusion joining process. The invention is also directed to a method for manufacturing such an implant, wherein the method of manufacturing allows the simultaneous coating of a plurality of implants (1) under “mild” process conditions.

### FIELD OF THE INVENTION

The present invention is directed to an implant according to the preamble of claim 1, and to a method of manufacturing such an implant. The implant according to the invention may be any type of a device implantable into a human or animal body, for example protheses such as heart flaps, joint protheses, vascular protheses (so-called “stents”), but also other implants such as cochlear implants.

### BACKGROUND OF THE INVENTION

An implant of the stent type is known from DE 199 16 086 B4. Such stents typically have a long, hollow cylindrical shape. They can be implanted into a blood vessel in order to maintain the vessel open. Frequently, stents are adapted to expand after having been inserted into the blood vessel, in order to remove a constriction or stenosis of the vessel. Vascular stents have achieved a considerable progress in the treatment of vascular diseases, but they are not free from problems and risks.

Restenosis, i.e. the repeated constriction of the vessel, still is the main problem in connection with stents. In the course of the development of medical technology, there were various attempts to prevent the neointimal proliferation (i.e. the in-growth) by coating the stents. This aim was not achieved by coating with gold; to the contrary: in the long-term progress, the in-growth rate was even increased. So far, coatings with silicon carbide and also carbon coatings did not show any clear results. Considerable improvements were brought about by the coating of stents with medicine such as Rapamycin (of Cordis Corporation) and Paclitaxel (of Boston Scientific Inc.). Meanwhile, corresponding studies show a considerable reduction of the restenosis rate, but not a complete absence of restenosis. Further, it is possible that the process of restenosis is simply delayed until a later time.

Stent implantation is not without risk. In particular, stent thromboses are dangerous, i.e. the formation of blood clots at the stent, which almost always lead to a myocardial infarction and are frequently deadly. In particular, in connection with medicine-coated stents, long-term studies revealed a large number of late thromboses.

Studies show that after placing a metal stent into a blood vessel, a cascade of reactions occurs. This cascade begins with the covering of the stent with a thin thrombosis layer, followed by a layer of smooth muscle cells, proliferation and finally the accumulation of an extracellular matrix, which is terminated by the formation of a complete endothelial layer on the stent. It is known that an excessive thrombis formation and the neointimal hyperplasia are the main sources of restenosis. Proliferation is considerably smaller or completely absent if the stent is rapidly endothelialized. On the contrary, an enlargement of the neointima is observed where the endothelialization is rather slow. Therefore, a rapid endothelialization appears advantageous in order to avoid or reduce the restenosis of stents.

There were already several attempts to accelerate the endothelialization of stents, for example by coating stents with a diamond-like carbon (c.f. U.S. Pat. No. 5,725,573) or with a porous layer of titanium or a titanium alloy (c.f. U.S. Pat. No. 5,690,670). Other attempts tried to accelerate endothelialization by the local or systemic addition of growth factors (Lindner V, Majack R A, Reidy M: “Basic fibroblast growth factor stimulates endothelial regrowth and proliferation in denuded arteries”; J Clin Invest 1990; 85: 2004-2008.; or Bjornsson T D, Dryjski M, Tluczek J, et al.: “Acidic fibroblast growth factor promotes vascular repair”;. Proc Natl Acad Sci USA 1991; 88: 8651-8655) by the local or systemic addition of medical substances (Guo J P, Panday M M, Consigny P M, Lefer A M: “Mechanisms of vascular preservation by a novel NO donor following rat carotid artery intimal injury”; Am J Physiol 1995; 269: H1122-H1131), or by substances for gene therapy (Asahara T, Chen D, Tsurumi Y, et al.: “Accelerated restitution of endothelial integrity and endothelium-dependent function after phVEGF165 gene transfer”; Circulation 1996; 94: 3291-3302). US 2005/0119723 A1 describes a stent with a porous coating. Particles of medical substances can be embedded into the nanopores.

Furthermore, there are suggestions to improve endothelialization by providing a suitable topography on the stent surface (Palmaz J C, Benson A, Sprague E A: “Influence of surface topography on endothelialization of intravascular metallic matedal”; J Vasc Intery Radiol 1999; 10: 439-444). Suggestions in this direction consist of providing grooves on a micrometer scale on the interior surface of the stent (U.S. Pat. No. 6,190,404) or in generating “controlled heterogenities” (US 2001/0001834), thereby allegedly accelerating endothelialization.

DE 199 16 086 B4 already mentioned above also describes a stent with a rough surface in order to improve the adhesion of the tissues cells. An intermediate layer of gold or platinum nanoparticles with cross-sections of 20 to 500 nm is provided on a main body of stainless steel, the intermediate layer defining the surface roughness. A thin outer layer of iridium-oxide or titanium-nitride is provided on the intermediate layer. In a similar manner, DE 199 21 088 A1 describes a stent with a coating of paramagnetic nanoscale particles. The paramagnetism of the particles shall be exploited for heating the stent, or for increasing the contrast in imaging magnetic resonance.

DE 103 61 941 A1 describes a magnesium coating for medical implants. This implant does not comprise nanoparticles, but rather microparticles. Further, these particles themselves do no constitute the coating of the implant, but they are merely embedded into a substrate material constituting the coating.

Temporary stents for non-vascular use are known from DE 103 57 742 A1. However, these temporary stents neither have a metallic coating on a metal main body, nor a coating of nanoparticles.

DE 102 43 101 A1 describes an open porous metal coating of joint implants. However, this document does not reveal anything about the material of the main body of the implant.

EP 1 679 088 A2 discloses joint protheses with a nanostructured coating. The joint protheses are made from a ceramic material.

WO 2004/110515 A1 describes the same stent as the above-mentioned DE 103 57 742 A1.

Chemical vapour deposition methods (CVD methods) for producing shape memory films, e.g. for stents, are known from US 2007/0061006 A1. Here, crystalline material with grain sizes in the sub-micrometer range result, but no nanoparticles are generated.

US 2006/0282172 A1 describes nanocrystalline protective coatings, e.g. for artificial hip joints. However, this protective coating—like in US 2007/0061006 A1—does not consist of single particles, but of a unitary layer.

In all conventional implants, it turned out to be disadvantageous that they are sometimes not stable enough in their use. This is important in particular with implants such as stents, which are intended to expand or otherwise change their shape after having been inserted into the body. Corrosion, as sometimes observed, or the complete chipping of parts of a coating are also disadvantageous.

### SUMMARY OF THE INVENTION

It is the object of the present invention to provide an implant which simultaneously supports a rapid endothelialization, and guarantees a high long-term stability.

This object is solved by an implant with the features of claim 1, or by a method for manufacturing an implant with the features of claim 12. Advantageous improvements of the invention are referred to in the dependent claims.

The implant of the present invention comprises a main body, and a coating of nanoparticles which is provided at least on portions of the surface of the main body. The material of the main body, and also a material of the nanoparticles (which may have dimensions from e.g. 10 to 500 nm) have a metal lattice structure. According to the invention, the lattice structure (i.e. the atomic or metal lattice structure) of the material of the nanoparticles is compatible with the lattice structure of the main body material in such a way that both materials are joinable or joined by diffusion of the materials (i.e. by a diffusion joining process, for example by an exchange of interstial atoms). In particular, this should allow for a joining of the main body and the coating not only at isolated locations, but in a laminar manner. In this way, several advantages are simultaneously achieved: by the coating of nanoparticles, the implant obtains an outer surface with a roughness which favors the adhesion of endothelial cells. The microscopic or nanoscopic intermediate spaces between the nanoparticles can be used for the anchoring of cells, e.g. in order to further support a rapid endothelialization, or to prevent the generation of thromboses. In addition, the compatibility of the lattice structure between the material of the base body and the nanoparticles results in a very strong attachment of the nanoparticle coating to the main body.

The reason for the extremely strong connection between the main body and the coating is that the nanoparticles are no longer (as in conventional implants) connected to the main body by relatively weak adhesion forces, but are very strongly connected with the main body by the exchange of lattice sites, as in diffusion welding. Even with the adverse environmental conditions present in the human or animal body, and possibly under the additional stress of an intended deformation, no parts of the coating can be lost from the implant. Instead, the exchange of lattice sites could even continue after inserting the implant under physiological conditions, thereby achieving an even stronger connection between the main body and the coating.

In the context of the present invention, the exchange of lattice sites between the material of the main body and the material of the nanoparticles means that a material connection results between the main body and the nanoparticles, in particular under formation of a common phase (from nanoparticles and main body) and a common, new surface. Due to this material connection, a (substitution) mixed crystal results which preferably does not show a sharp phase boundary anymore between the nanoparticle and the main body.

Due to the compatibility, i.e. the favorable ratio between the atomic lattice distance (lattice constant) of the main body material and the atomic lattice distance of the coating, a strong connection is achievable between the main body and the coating, since a diffusion of atoms into a substitution mix crystal for the diffusion of single material atoms onto the intermediate lattice sites of the other material is facilitated. The possibility of such a diffusion is the prerequisite for a close material connection, i.e. for a diffusion welding, or more specific: a diffusion joining (the term “joining” is more appropriate than “welding”, since the invented method does not require the temperatures and pressures necessary for welding). The method of the invention further allows a very high freedom of geometry and, simultaneously, considerably low-cost process conditions.

Depending on the material and the surface topography, the nanoparticle coating can also reduce the friction between the implant (e.g. a stent) and a delivery system which delivers the implant into the body. This would be particularly advantageous in connection with long “peripheral” stents, i.e. stents for use in the peripheral areas of the blood circuit, which may often be delivered only under considerable difficulties.

If additionally the material of the main body and the material of the nanoparticles substantially (i.e. with a maximum deviation of approximately 5%) have the same electrochemical potential, this guarantees that a corrosion between coating and main body is effectively suppressed. This is will further improve the long-term stability of the implant. Passivating intermediate layers can be omitted.

In a special embodiment of the invention, the main body and the nanoparticles of the coating are made from the same material. This will guarantee a complete adaptation of the lattice structures and, hence, a particularly strong, stable connection of the coating to the main body.

If the implant is intended to be deformable, for example an expandable stent, shape memory materials such as a nickel titanium alloy are particularly advantageous. If the material of the main body is such a nickel titanium alloy, the nanoparticles can comprise one or several of the following materials, which result in a very good adaptation of the metal lattice structures:
a) titanium (Ti),
b) nickel titanium (NiTi),
c) Ni(x)Ti(y), where x and y together yield (almost) 1,
d) NiTiX, i.e. nickel titanium with an insertion or an alloy partner “X”, for example as NiTiAg with an anti-bacterially active silver additive,
e) TiOx,
f) TiOx(OH)y, where x and y together yield (almost) 1, or
g) Ni(x)Ti(y)O(z)H(n), where x, y, z and n together yield (almost) 1.

An adaptation of the lattice structures is advantageous in particular for such pseudoelastically deformable materials. It guarantees that the pseudoelasticity of the implant or the shape memory actor effect of the main body does not (or hardly) change by providing the coating. Since the main body and the coating deform in the same manner and are additionally very strongly connected, a losing of the coating can effectively be prevented.

The material of the main body, however, could also be a Co—Cr alloy (for example one of the stent alloys L-605 or MP-35N) or a stainless steel (for example 316-L). In these cases, e.g. chrome alloys or biocompatible steels or iron alloys are suited as diffusion compatible materials for the nanoparticle coating.

It is conceivable that the main body has an outer surface and an inner surface, and that the coating is only provided on one of the two surfaces, in order to thereby intendedly favor the adhesion of new endothelial cells on this coated surface.

Alternatively, however, the coating could be provided both on the outer surface and on the inner surface of the main body, e.g. if an adhesion of tissue cells on both surfaces is considered advantageous.

Surprisingly, it turned out that the coating does not have to be provided on the complete surface of the main body in order to allow for a rapid endothelialization. Rather, it will suffice if the nanoparticle coating is provided on 30-70% of the surface of the main body, preferably on approximately 50%. Tissue cells will adhere to the coated area. These tissue cells will subsequently serve as a “nucleus” for the adhesion of further tissue cells. Hence, even with a partial but thereby cheaper coating of the main body, a very rapid endothelialization can be achieved.

It has also turned out that the adhesion of tissue cells is further favored if the nanoparticles are inhomogenously dispersed on the main body.

The nanoparticle coating does not necessarily have to constitute the outer surface of the implant, but the nanoparticle coating could also be provided with a further cover or overlay from one or several layers.

The invention is also directed to a method of manufacturing an implant, wherein a nanoparticle coating is provided on a main body, and the metal lattice structures of the material of the main body and the material of the nanoparticles are mutually compatible. In this way, an extremely strong, durable connection between the main body and the coating results.

As examples, the following techniques are available for providing the nanoparticles on the main body: coating in an immersion bath of a colloidal nanoparticle suspension, a spray deposition and/or an electrophoretic deposition of the nanoparticles on the main body. Very advantageous is the high freedom of the main body geometry, which merely depends on the degree of wetting with a fluid, and which is free from shading effects. Even peripheral stents with very small diameters or stents with a high portion of material (i.e. only few cut-outs) can be coated without any disadvantages.

According to the invention, the provision of the nanoparticles on the main body can occur at a temperature of 15° C. until 40° C., preferably at 20° C. to 30° C. This offers several advantages: a process at room temperature is comparatively cheap and—due to avoiding heat-up times—can be performed very rapidly. In addition, the deposition and the strong attachment of the nanoparticles occurs in a temperature range which the implant is also exposed to after having been inserted into the body.

Preferably, the provision of the nanoparticles according to the invention is performed under normal pressure, which results in both cost advantages and process technical advantages in comparison to vacuum coating methods.

In a particularly advantageous variant of the invention, nanoparticles may be connected with the material of the main body by a diffusion process, in particular a diffusion connection. The resulting connection between the nanoparticle coating and the main body is extremely strong due to the exchange of lattice sites.

It is conceivable that parts of the surface of the main body are covered prior to and/or during the provision of nanoparticles, in order not to be coated with nanoparticles. Such uncoated areas can e.g. be used for better handling of the implant, or for marking certain areas of the implant. It is also possible with a main body having an outer and an inner surface to only cover one of the two surfaces, such that the coating is obtained only on the other of the two surfaces.

In order to cover the main body, a covering could be used that is removed again after providing the nanoparticles, for example a layer from a soluble polymer, or an elastic tube.

The nanoparticles can be obtained by different methods. It turned out to be particularly advantageous to obtain the nanoparticles by removal or abrasion from a substrate by means of a pulsed laser, in particular a short-pulse laser or an ultra-short-pulse laser, since the size of the nanoparticles can be precisely adjusted by suitably choosing the laser parameters and the focusing parameters. Due to the extremely short interaction times of the laser (with pulse durations in the nano-, pico- or femtosecond range), the abrasion of the nanoparticles from a substrate by means of a pulse laser avoids the delivery of heat into the substrate or the particles. Consequently, the atomic lattice structure of the substrate material is conserved, and a previously adjusted compatibility of the atomic lattice structures of the substrate material and the material of the main body is transmitted in an ideal way onto the nanoparticles. Hence, the nanoparticles obtained in this way are particularly suited for the desired diffusion connection process.

It is advantageous if the substrate is from the same material as the main body, or is obtained by laser abrasion from the same material. In particular, the main body itself, or similar main body, could be used as the substrate from which the nanoparticles are obtained. This will guarantee that the nanoparticles consist of the same material as the main body, such that they also have the identical metal lattice structure as the material of the main body, so that the lattice structures are diffusion compatible in an optimal way.

Subsequent to providing the nanoparticle coating, the coating could also be provided with a thin mono- or multi-layer overlay.

A great advantage of the method of the present invention is that it allows the simultaneous parallel coating of a plurality of main bodies with nanoparticles. If e.g. several tens, several hundred or even several thousand implants are simultaneously coated, the costs per piece can be reduced enormously. At the same time, the parallel manufacturing offers the advantage that the implants are coated under the same conditions, such that the characteristics of the coating should also be identical for all implants. For quality checks, it is therefore sufficient to watch and document the manufacturing process for individual of the products manufactured in parallel.

### BRIEF DESCRIPTION OF THE DRAWINGS

In the following, a presently preferred embodiment of the invention is described in connection with the attached drawings.

FIG. 1 shows an embodiment of an implant according to the invention in the form of a stent, and

FIG. 2 shows a vertical section through the stent shown in FIG. 1 at the position labeled II-II in FIG. 1.

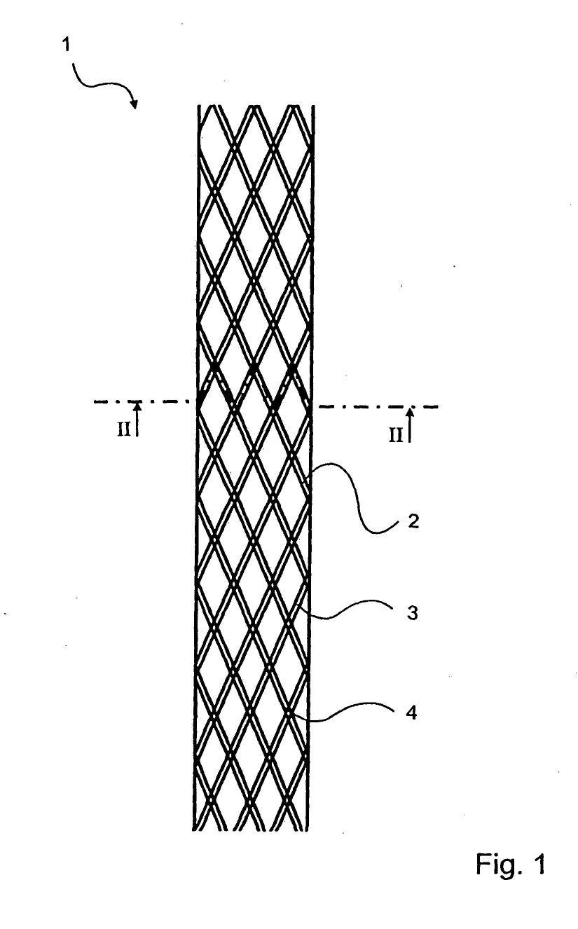

FIG. 1 is a top view onto an embodiment of an implant 1 according to the invention, which here is a stent for insertion into a blood vessel. The stent 1 comprises a main body 2, which has a hollow cylindrical, net-like shape. The net-like shape of the main body 2 in this case results from two mutually inclined groups of net strands 3, wherein the strands 3 of each group mutually run in parallel and are connected at nodes 4 with the strands 3 of the other group. The complete main body 2 of the implant can be made unitary.

In the present embodiment, the main body 2 is made from a metallic shape memory material, which consequently has an internal metal lattice structure. The material could in particular be a nickel titanium alloy (NiTi or “nitinol” respectively). This shape memory material allows the stent to deform after insertion into the body of the patient, such that the interior diameter of the hollow cylinder is enlarged and the stent more strongly adapts to the walls of a blood vessel.

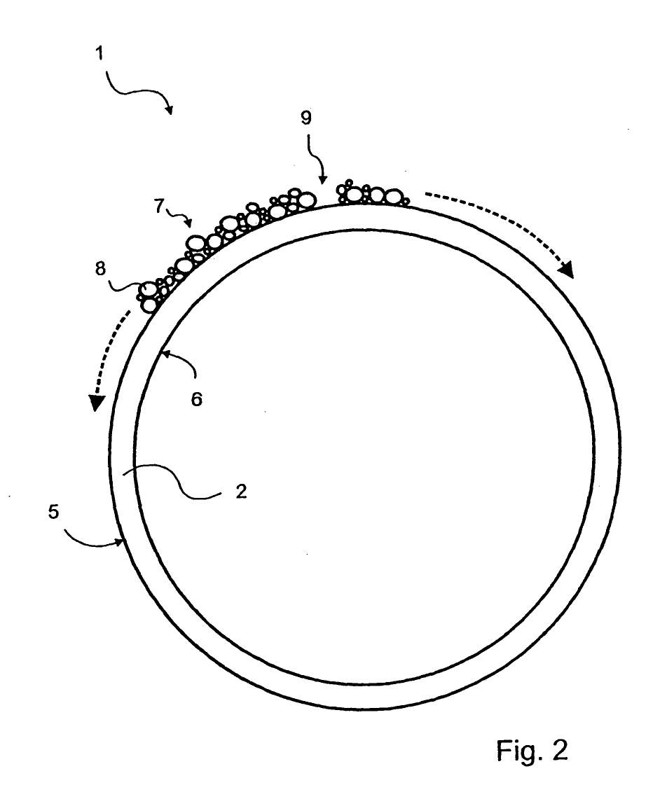

FIG. 2 shows a vertical section through the stent 1 at the position labeled II-II in FIG. 1. Hence, this section follows the cause of the net-strands 3 of the main body 2.

Due to its hollow cylindrical shape, the main body 2 has an outer surface 5 and an inner surface 6. While the inner surface 6 of the shown embodiment is free from a coating, a coating 7 is provided on the outer surface 5. Typically, however, also (or even entirely) the inner surface 6, i.e. the surface of the stent directed to the blood stream, is provided with a coating 7. For the embodiment of the implant as a stent 1, an interior coating can even be particularly advantageous. At the ends or side edges of the stent 1, the coating 7 can also be provided in order to accelerate the endothelialization at these positions. The coating 7 consists of nanoparticles 8, i.e. of particles with sizes of less than 1 μm. In particular, the nanoparticles 8 may have similar or different diameters in the range from approximately 10 nm to 500 nm.

According to the invention, the material from which at least a portion of the nanoparticles 8 is made has a metal lattice structure which is substantially compatible, if not even identical to the metal lattice structure of the material of the main body 2. If the main body 2 is made from nitinol, the nanoparticles might e.g. comprise titanium (Ti), nickel titanium (NiTi), Ni(x)Ti(y), TiOx, TiOx(OH)y, Ni(x)Ti(y)O(z)H(n) or combinations of these materials, as the metal lattice structures of these materials substantially correspond to the metal lattice structure of NiTi. Preferably, the metal lattice structures should be compatible in such a way that, during attachment of the nanoparticles to the main body, an exchange of lattice sites is possible.

The coating 7 has a thickness of approximately 20 nm to 500 nm. However, it could also be higher, for example up to 1.0 or 1.5 μm. The two arrows indicate that the coating could extend round the complete main body 2, even if only a small section of the coating 7 is shown. However, uncoated areas or “coating gaps” 9 are provided between coated portions of the main body. The “coating gaps” 9 result in areas which are covered during deposition of the nanoparticles 8.

The rough surface of the implant 1 offers ideal conditions for the attachment of endothelial cells, while the undesired attachment of smooth muscle cells is not supported. Although not shown in FIG. 2, the outer surface of the coating 7 can optionally be provided with an additional mono- or multi-layer film-like overlay, which does not substantially alter the surface topography of the implant. The overlay could comprise medical or other substances which favor the attachment of certain cell types.

The implant 1 is manufactured by initially forming the main body 2, and separately thereto generating the nanoparticles 8. Preferably, the nanoparticles 8 are generated by abrasion or ablation from a substrate or base material by means of a short-pulse laser or ultra-short-pulse laser. Via the chosen parameters of the laser, in particular the pulse energy and the pulse duration, the size of the nanoparticles 8 ablated from the substrate can be adjusted. This way of nanoparticle generation is described in the articles “Continuous production and online-characterization of nanoparticles from ultrafast laser ablation and a laser cracking” of S. Barcikowski, et. al., Proceedings of 23^{rd} International Conference on Applications of Lasers and Electro-Optics ICALEO 2005, October 31 to November 3, Miami, Fla., USA, pages 375 to 384, as well as “Properties of nanoparticles generated during femtosecond laser machining in air and water” of S. Barcikowski et. al., Appl. Phys. A 87, 47-55 (2007). It has turned out that the laser ablation mentioned above is also suited for generating nanoparticles from metal shape memory materials.

In particular, the laser abrasion from the substrate can be conducted in a fluid environment, as in this way the nanoparticles are immediately dispersed colloidally stabilized after their generation, such that they are maintained as “individual” nanoparticles without grouping themselves to larger agglomerates. The fluid could either be directly used for the coating, provided with an additive, or subsequently be exchanged. Further, it is possible to provide additional alloy partner for the ablated material in order to maintain the stoichiometry, if, otherwise, one of the alloy partners is not present in the nanoparticles to a sufficient degree.

Subsequently, the nanoparticles are deposited onto the main body 2. The deposition of the nanoparticles 8 can be performed after an optional (electro) polishing of the main body 2. Preferably, the deposition is performed under room temperature, for example by electrophoretic deposition. Due to the compatibility of the metal lattice structures, the deposited nanoparticles 8 exchange lattice sites with the material of the main body 2. This diffusion connection process results in an extremely strong connection between the coating 7 and the main body 2. This connection is so strong that not even during an expansion of the stent 1 (stent dilatation) portions of the coating 7 are lost. In conclusion, this process results in an implant 1 with a particularly good long-term stability.

For certain materials and for certain applications, the exchange of atoms in the crystal lattice obtained after depositing the nanoparticles 8 on the main body 2 at room temperature is already sufficient for achieving a strong attachment of the nanoparticles 8 on the main body 2. An even stronger attachment of the nanoparticles 8 can be achieved if the temperature of the implant 1 is raised after deposition of the nanoparticles 8 on the main body 2 onto a diffusion connection temperature which is higher than room temperature, and at which consequently the main portion of the diffusion connection process is performed. Since at the higher diffusion connection temperature more atoms of the lattice structure of the nanoparticles exchange their site with atoms of the lattice structure of the main body 2, the nanoparticles 8 are subsequently connected considerably stronger with the main body 2.

The subsequent processing after the deposition of the nanoparticles 8 is preferably performed at a (diffusion joining) temperature and at a pressure which, in the pressure-temperature-phase diagram are located below the melting point or the sublimation point of the material of the nanoparticles 8 and the main body 2. This will guarantee that the main body 2 and the nanoparticles 8 maintain their macroscopic structure. Preferably, the temperature of the diffusion joining process is in the range of 60% to 80% of the melting temperature of the material, or slightly below, in order to minimize the grain growth. For NiTi, the diffusion joining process can preferably be performed below 300° C., preferably below 150° C. For steel stents or other metal stent materials, other diffusion joining temperatures can be advantageous.

Starting from the above-described embodiment, the invention could be modified in several aspects. It should be emphasized that the invention is not restricted to stents, but that also other implants such as heart flaps, carrier structures for heart flaps, blood filters, closure devices, vascular connectors, stent grafts, etc. could be coated with a corresponding coating of nanoparticles. If the invention is applied on stents, it is not at all compulsory that the stent should have the shape shown merely as an example in FIG. 1. Rather, the shape of the stents could be considerably more complex. Similarly, it is not necessary that the main body of the stent must be made from a shape memory material. Conceivable are also stents from steel or other materials which are expanded by a balloon inserted into their interior. Medical substances, genes, growth factors or other could be attached to the nanoparticle coating 7 or inserted into the pores of the coating 7, these substances having a positive effect on the environment of the implant 1. Even if the deposition of the nanoparticles 8 at room temperature is advantageous from a process technical point of view, it could also be performed at other temperatures (and pressures), for example at 4° C. to 100° C.

1-24. (canceled)

25. Method for manufacturing an implant with the following steps:
a) producing a main body of the implant from a material with a metal lattice structure,
b) separately from manufacturing the main body, generating a plurality of nanoparticles from at least one material which also comprises a metal lattice structure, wherein the metal lattice structures of the material of the main body and the material of the nanoparticles are mutually compatible in such a way that, after depositing the nanoparticles as a coating on at least a portion of the surface of the main body, the nanoparticles join with the material of the main body by a diffusion joining process, during which atoms of the lattice structure of the nanoparticles exchange their site with atoms of the lattice structure of the main body,
c) wherein the deposition of the nanoparticles on the main body is performed at a temperature of 15° C. to 40° C., and at normal pressure,
and wherein the deposition of the nanoparticles on the main body comprises an electrophoretic deposition of the nanoparticles on the main body.

26. Method according to claim 25, wherein the deposition of the nanoparticles on the main body comprises a coating in an immersion bath and/or a spray deposition of the nanoparticles on the main body.

27. Method according to claim 25, wherein the deposition of the nanoparticles on the main body is performed at a temperature of 20° C. to 30° C.

28. Method according to claim 25, wherein portions of the surface of the main body are covered prior to and/or during the deposition of the nanoparticles, in order to locally prevent a coating with nanoparticles.

29. Method according to claim 28, wherein the covering is performed by means of a removable cover.

30. Method according to claim 25, wherein the nanoparticles are generated by abrasion from a substrate by means of a pulsed laser, in particular a short-pulse laser or ultra-short-pulse laser.

31. Method according to claim 30, wherein the laser abrasion is performed within a fluid.

32. Method according to claim 30, wherein the substrate consists of the same material as the main body.

33. Method according to claim 25, wherein the coating is provided with at least one additional coating.

34. Method according to claim 33, wherein a plurality of main bodies are simultaneously provided with a coating of nanoparticles.

35. Method according to claim 25, wherein the diffusion joining process is performed at a higher temperature than the deposition of nanoparticles on the main body.

36. Method according to claim 25, wherein the diffusion joining process is performed at a temperature and a pressure which are situated below the melting or sublimation point of the material of the nanoparticles and the material of the main body in the pressure-temperature phase diagram.

37. Method according to claim 36, wherein the diffusion joining process is performed at a temperature which—measured in degrees Celsius—is lower than 80% of the melting temperature of the material of the nanoparticles or the material of the main body, preferably lower than 60% of the melting temperature.

38. Implant, manufactured by a method according to claim 25, with a main body having a surface, and with a coating from nanoparticles being provided at least in portions of the surface of the main body, wherein the main body is made from a material with a metal lattice structure, and the nanoparticles of the coating comprise a material which also has a metal lattice structure,
wherein the lattice structure of the material of the nanoparticles is compatible to the lattice structure of the material of the main body in such a way that both materials are joined to each other by a diffusion joining process, during which atoms of the lattice structure of the nanoparticles have exchanged their site with atoms of the lattice structure of the main body.

39. Implant according to claim 38, wherein the material of the main body and the material of the nanoparticles substantially have the same electrochemical potential.

40. Implant according to claim 38, wherein the main body and the nanoparticles of the coating are made from the same material.

41. Implant according to claim 38, wherein the material of the main body is a nickel titanium alloy, and the nanoparticles comprise one or several of the following materials:
a) titanium (Ti),
b) nickel titanium (NiTi),
c) Ni(x)Ti(y), wherein x and y together yield 1,
d) NiTiX,
e) TiOx,
f) TiOx(OH)y, wherein x and y together yield 1, or
g) Ni(x)Ti(y)0(z)H(n), wherein x, y, z and n together yield 1.

42. Implant according to claim 38, wherein the material of the main body is a Co—Cr alloy.

43. Implant according to claim 38, wherein the material of the main body is a stainless steel.

44. Implant according to claim 38, wherein the main body comprises an outer surface and an inner surface, and the coating is only provided on one of the two surfaces.

45. Implant according to claim 38, wherein the main body comprises an outer surface and an inner surface, and the coating is provided on both surfaces.

46. Implant according to claim 38, wherein the coating is provided on 30% to 70% of the surface of the main body, preferably on approximately 50%.

47. Implant according to claim 38, wherein the nanoparticles are inhomogenously distributed on the main body.

48. Implant according to claim 38, wherein the coating is provided with an overlay.
